(19) United States
(12) Patent Application Publication
(10) Pub. No.: US 2025/0271411 A1
(43) Pub. Date: Aug. 28, 2025

(54) Installation method for real-time fruit, vegetable, and nuts monitoring

(71) Applicants: Mason Erkelens, Panorama SA (AU); Amanda Jane Mader, Panorama SA (AU)

(72) Inventors: Mason Erkelens, Panorama SA (AU); Amanda Jane Mader, Panorama SA (AU)

(73) Assignee: Ripen Tech Pty Ltd, Panorama SA (AU)

(21) Appl. No.: 19/054,858

(22) Filed: Feb. 16, 2025

(30) Foreign Application Priority Data
Feb. 27, 2024 (AU) AU2024900385

(51) Int. Cl. G01N 33/02; G01G 3/00; G01G 19/00; G01G 23/18; G01N 33/00

(52) U.S. Cl. CPC G01N 33/025; G01G 3/00; G01G 19/00; G01G 23/18; G01N 33/0098

(57) ABSTRACT

Real time plant product monitoring is important for growers in evaluating the current crop weights and irrigation planning. Plant products respond to the amount of water, heat, or soil conditions can impact the plants products weights. As the user monitors the data, they can determine potential issues with their crop in real-time and develop a plan to respond to issues.

### BACKGROUND INFORMATION

Real-time fruit, vegetable, nuts weight monitoring can now be undertaken with a variety of different electronic means. Monitoring the fruit, vegetable, or nuts in real-time can assist growers with key information on how their crop in respond to environmental or irrigation practices.

The common practice in how this would traditionally be undertaken is a grower will visit the field and take samples periodically and weight it. This is a labour-intensive task that also destroys the crops to obtain the fruit for measurement.

This method is also subjective to variation due to the diurnal changes in weight, this variation in daily weight can be up to 30%. This variation can cause issues when estimating the current yield of a vineyard.

To date users install real-time weight sensors by connecting fruit to a load cell with a string, and the string is tensioned. However, the tension to the connector is not known as it is difficult to differentiate the plant and the product of the plant.

Also, users may not know how to interpret the data, such as if the recorded weight of one fruit or vegetable represents the minimum, average, or maximum within their crop.

Within this patent, we will provide a solution to the issues discussed above.

### SUMMARY OF THE INVENTION

In a first aspect the invention there is a proposed a method for real-time measurement of a plant product while it remains attached to a plant, the method comprising selecting a representative sample of a plant product and measuring its weight to determine a reference weight threshold; attaching a sensor to the plant product, wherein the sensor captures weight data; adjusting the attachment of the sensor to the plant product until the sensor measures a weight within a predefined tolerance of the reference weight threshold; and recording and transmitting the weight data of the plant product to a remote device for monitoring.

In preference wherein the sensor comprises one or more of the following: a load cell, camera, dendrometer, or a combination thereof.

In preference wherein the threshold is determined based on a minimum, maximum, average, mean, or standard deviation of sample weights.

In preference wherein the plant product comprises a fruit, vegetable, nut, seed, or other agricultural or botanical produce.

In preference the sensor further comprises one or more of a temperature sensor, humidity sensor, optical sensor, infrared sensor, or gas sensor for monitoring environmental and physiological conditions.

In preference the sensor is wirelessly connected to a remote device using Bluetooth, Wi-Fi, LoRa, or another wireless communication protocol for real-time data transmission.

In preference the wherein the sensor continuously records weight changes over time and generates a growth trend analysis to predict plant product development.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows.

FIG. 1 is an illustration of the device connected to a product wherein it is using an external load cell.

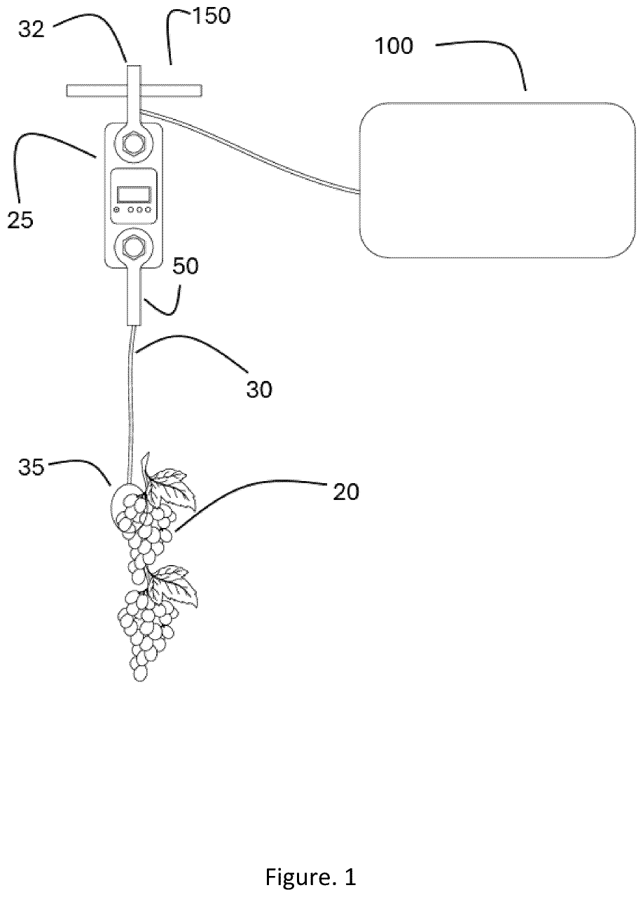

FIG. 2 is an illustration of the device wherein it contains an internal load cell.

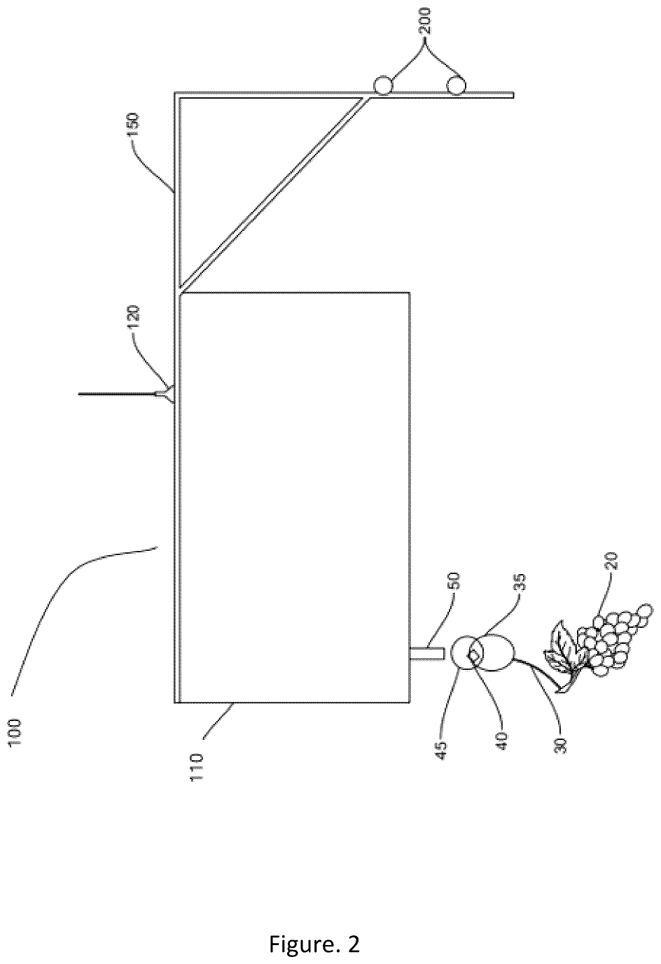

### DETAILED DESCRIPTION

An installation method wherein a sample set is taken, the samples are weighed, a sensor is installed, and a load is placed onto the sensor to match the sample set taken either within the weights taken or an average.

This installation method can be applied to any product a plant produces.

The plant is defined as any component of a plant including but not limited to the trunk, branch, leaf, stem, rachis, and roots. The plant is not limited to any species.

A product 20 can include but not limited to apples, pears, mangos, avocados, grapes, chillies, capsicums, eggplants, macadamias, stone fruit, melons, flowers, hops, squash, potatoes, cherries, plums, berries, and any other edible and non-edible product from a plant.

The plant can be in a crop, a crop can be defined as but not limited to a crop, orchard, scrub, garden, greenhouse, vineyard, indoor growth area, vertical farm, or a location wherein plants are used to grow products.

Firstly, the user would like to monitor a crop product of interest. A crop containing a product of interest is identified that is being grown on a plant.

The plant containing the product 20 can be but is not limited to free-standing, trellised, underground, vertical farming, or any other farming method used.

Once the product 20 has been identified, a sample is taken of the product 20. The samples of the product 20 are taken by but not limited to cutting the product 20 off the plant or pulling it. The sample can consist of but is not limited to some or none of the plant.

The duration of when the product 20 sample is taken and recorded is not a limiting factor, though ideally it would be undertaken as soon as the product is taken from the plant.

The user can also opt on how much of the product 20 and plant they wish to include in their sampling.

For example, with grapes, the user may opt to remove all the steam and just weigh the berries. The sampling method used by the user will be recorded, as the final weights are obtained by the device further in the process will represent the initial sample method used.

The number of product 20 samples of the product 20 taken can be but not limited to 1 to 4000 products 20.

The location of the product 20 samples can also be used, for example, the user may take samples from one region, part, or section of a plant.

The selection of the samples can be in correspondence with an area of interest for the user.

For example, the user would like to record the weights of the south side of the plants in comparison to the north side. The recorded weights can then be used as the desired weights.

Once the product 20 samples are taken, they are weighed and recorded.

The product 20 weights are then analysed by but not limited to minimum weight, average weight, mean weight, maximum weight, and standard deviation weights.

Once the analysis of the product 20 sample weights are undertaken, they are recorded and can be defined as but not limited to, desired weight, target weight, minimum weight, average weight, mean weight, crop weight, product weight, maximum weight, mean weight, standard weight, sample weight, or weight.

A device 100 that can monitor the product 20 weight is then installed.

The device 100 can measure the weight of the product 20 via a sensor 25, the sensors 25 can be but are not limited to a load cell, tension cell, strain sensor, hydraulic sensor, pneumatic sensor, photo image to estimate weight, means to measure the diameter and convert that into a weight, or any other means to measure weight.

The sensor 25 can be connected to the product 20 directly or via the connector 30, this can be undertaken by but not limited to directly to the product 20, or close to the product 20, partially connected to the product 20, multiple connections to the product 20, a branch supporting the product 20, or the whole plant.

For example, the sensor 25 is connected to the connector 30, which is connected to a bunch of grapes wherein the connector is looped around the first few berries of the grape bunch. As another example the connector 30 is connected to the bottom and top of the grape bunch, this example could also include a net to support the grape bunch.

The sensors 25 are not limited to where the sensors 25 or weight recorded device are located, the sensors 25 can be but not limited to internal or externally located.

If the sensor 25 is externally located, it can have an upper connector 32 which connects the sensor 25 to a fixture 150.

For example, the external sensor 25 is fixed to a trellis wire which is used as a fixture 150.

Another example is wherein the external sensor 25 upper connector is connected to a post as a fixture 150, and the sensor 25 is then supported by the upper connector 32. The sensor 25 is then connected to a product 20 with a connector 30. The adjustable screw 50 is connected to the connector 30 to adjust the length of the connector 30.

The externally located sensors 25 can be but not limited to 1 cm to 5 km in distance.

For example, the sensor may communicate to the device 100 wirelessly allowing the user to place the sensors 25 throughout the orchard.

The product can be attached to the sensor 25 with a connection 30, the connection 30 can be but not limited to a cord, net, string, platform, callipers, arms, claw, cloth, cup, bucket, bag, or any other means of connection.

Photo images can be used to measure the weight of a product 20 by taking an image and comparing it against other similar products 20 to estimate the weight. Within these specifications, this can be used as an alternative to a load cell as discussed in the examples.

The diameter of a product 20 can be used to determine the weight of a product 20, this can be achieved by measuring the diameter and correlating it with the weight. As the diameter changes, the weight is estimated.

The diameter sensor can be but is not limited to a dendrometer, callipers, stretch sensor, laser, ultrasound, computer vision, or an electronic motorised means that is measured over time.

Within these specifications, the diameter or a photo of the product can be used as an alternative to a load cell as discussed in the examples. However, the methods to take an initial sample set and calibrate it accordingly can still be applied.

The device is installed near a product 20 or products 20 of interest by attaching it to a fixture 150, a fixture 150 can be but is not limited to a post, star picket, wall, the plant itself, or any other means to secure it.

The device 100 enclosure 110 can be attached to the fixture 200 but not limited to cable ties, screws, bolts, wire, string, welding, or pop rivets.

The fixture 150 will support an enclosure, wherein the enclosure 110 can be but not limited to being mounted on the top, bottom, or sides of the fixing.

The enclosure 110 can be but is not limited to a box, housing, container, ball, custom shape, or tube.

The enclosure 110 contained the device which is used to house the device 100. The device 100 contains all the components required to capture the plant's product 20 weights.

The enclosure 100 can also be directly attached to but not limited to a wall, the plant, trellis, wire, cord, rope, pole, or post.

The enclosure 110 can also have a fixing 150 integrated into it, for example, the enclosure 110 can have but is not limited to a bracket, holes, or a means for a screw, bolt, or strap used to attach it to a structure of interest.

The fixing 150 can be but not limited to a bracket, shelf, bolt, ledge, or any other form of supporting structure.

The enclosure 110 can also be directly fixed to a plant either by but not limited to, cables, ties, string, cords, adhesives, pins, screws, bolts, or any other suitable form to affix it.

The enclosure 110 contains the main components of the device or anything the user would like the enclosure to retain.

The enclosure 110 can contain a user interface, wherein the user interface can be but not limited to screen, a button, a switch, a dial, a port for an external device to be plugged into, or any other means for a user to communicate with the device.

The main components of the device 100 can consist of but not limited to, a CPU, microcontroller, power management, load cell amplifier, visual screen, battery, cellular communications, communication module, solar charging management board, or any other devices required to measure weight and communicate it.

If required, the main components can be also located on the outside of the enclosure 110 in other enclosures such as but not limited to external batteries, external solar modules, external power modules, external antennas 120, external communication modules, external sensor boards, and external sensor amplifier.

The device 100 can also consist of other sensors such as but not limited to temperature, humidity, spectrometry, light, pressure, gasses, flame, UV, cameras, photonics, fibre, and movement, rain fall, soil moisture, radiation, and tension.

The device 100 can contain a variety of power options such as but not limited a battery or solar.

To assist battery life, the device can use but is not limited to sleep functions, low power modes, or shutting off certain components.

Once the device 100 is mounted, the device is connected to the product of interest. Alternatively, is a photo image is used to measure the product of interest it will be positioned so it can acquire the photos of the product 20 accordingly.

The photo methods can also use but not limited to a background with reference points to assist measurements.

The number of sensors 25 or photo acquiring means a device can be but is not limited to 1 to 10000.

If a weight sensor 25 is being used, the sensor 25 can consist of an adjuster 50 wherein it can be adjusted length wise, the adjuster can be but is not limited to an adjustable screw, a slider, an interference fit pipe, and any other means to adjust the length.

For example, if the device 100 is using a load cell to monitor the fruit weight, the load cell is connected to the product 20 using a connector 30, the connector 30 is connected to a connector ring 35, which is connected to a swivel 40, which is connected to a sensor ring 45, that is connected to an adjustable screw 50. This example does not limit the means a product 20 can be connect to the sensor 25, any means can be used to connect the product 20 to the sensor 25.

The adjuster 50 can be but not limited to string, wire, bolt, screw, solid rod, cord, spring, or any other means that can support weight.

The adjuster 50 is preferably adjustable.

The connector 30 can be connected to the sensor 25, the connector 30 can be but not limited to an eye bolt, through a hole, an interference fit, a fixing bolt to retain the connector, a plug, or any other means that can fix the connector to the load cell.

Once the sensor 25 is connected to the product 20 by a connector 30, the connector 30 is torqued to wherein the sensor 25 reads a desired weight. The connector 30 is torqued by but not limited to adjusting the length of the adjuster 50, or adjusting the length of the connector 30, or adjusting the plant position, or adjusting the product 20 position.

The desired weight will be set as desired by the user, for example, it can be but not limited to the product 20 samples minimum weight, average weight, mean weight, maximum weight, or any weight in between the minimum or maximum weight.

As the connector 30 is attached to the device 100, the plant can be fixed to but not limited to trellis, post, wall, or anything to prevent the plant and product 20 from moving. This can also be undertake prior to the connector 30 being attached to the plant product 20 or after it is attached to the product 20.

Once the desired 100 weight is achieved the connector 30 can be fixed to retain the desired torque.

The connector 30 can be fixed by but not limited to using a grub screw, interference fit, a fixing screw, knots, or any other means to prevent movement.

As the user in installing the device 100 and connecting a sensor 25 to the product 20, the desired weight can be within the minimum to maximum weight, or outside of those weights if required. The user can set the tolerance in accordance with their needs. For example, if a minimum weight is recorded, the user can set the connector's tension up to but not limited to 0 to 10000 grams of the desired weight.

For example, if the desired weight is 100 grams as the minimum weight, the user can tension the connector until the device reads anywhere between 30 grams to 2000 grams if required.

Another example, if the desired weight is 100 grams, the user can tension the wire to more weight than 100 grams, and then trim the fruit or plant until the desired weight has been reached.

The user can monitor the weight on the device 100 or remotely via but not limited to a user interface, a phone, a computer, dial, visual gauge, cloud service, dashboard.

The user can receive the weight data remotely or view it on the device itself.

The device 100 can contain two modes, a setup mode wherein the device will provide a high rate of weight reads, and an operational mode wherein the device will only send data during a set period of time as desired by the user.

For example, during setup, the device 100 takes a read every 1 second, once set up the device 100 will send a weight to the user every hour to preserve the battery and reduce data usage.

The user can select the device 100 to send a signal between 1 second to 1 year, the user will select the amount of data to be received in accordance to how much battery and data they would like to use. The more frequent the weight reading from the device 100 the more data and power used.

The device 100 can also consist of an over the air programming, wherein the devices firmware can be updated remotely.

The desired weight can be but not limited to the minimum, maximum, average, mean, a standard deviation, or any weight they desire.

The device 100 will communicate the weight of the product 20 to the user and if required store the weight data on the device 100 via memory storage.

The means of communication 200 the device 100 uses can be but not limited to cellular, LoRa, Sigfox, Bluetooth, Wi-Fi, radio, satellite, or any other form of communication. The device is not limited to its means of communication and can be adapted accordingly to communicate the weight readings to the users by any form.

The memory storage on the device can be but not limited to a SD card, hard drive, USB, EPROM, or any other means of storage for data on an electronic device.

The communication module in the device can be modular wherein it can be replaced if needed.

The device 100 will communicate the weight to the user on a but not limited to 0.1 second- or 1-year interval, the user will select a suitable duration as they require.

The obtained weight on the device 100 which is connected to the product 20 will be used to represent the desired weight for the crop.

For example, the user installs the device 100, as the user is connecting the load cell to the product 20 via a cord, the user is monitoring the weight the device 100 is reading, once the user observed the desired weight is reached, or is close to being reached he fixes the connector 30. If the average weight of the sample is set on the sensor, the user can interpret that data as the crops whole average weight, as the product 20 weight increases over time and is recorded by the device 100 and sent to the user, the user can interpret the recorded weight as the estimated or exact average weight of the crop.

The grower would like to know how the south side of the tree products 20 respond to irrigation. The user samples the apples only on the south side of the trees. The user then records the weights of the samples taken and calculates the minimum, average, and maximum weight. The user then installs the device 100 on a nearby post near an apple tree. The user then connects the sensor 25 which is a load cell on the device 100 to an apple 20 that visually represents the minimum, average, and maximum size apples. The user tensions the connector 30 cables connecting the load cells until the representative apple is close to or matches the weight it is representing. For example, if the minimum weight is 50 grams, and the user is tensioning the connector 30 to the apple until it represents the minimum weight apples within the users tolerances, the user will keep tensioning the connector 30 cable until the device reads close to or matches 50 grams. Once the user is content with the tension on the connect 30 the device 100 is reading, the connectors 30 is fixed and locked so the tension can no longer change. As the apple grow in weight, the user can track the crops minimum, average, and maximum weights. The weights provided to the user by the device 100 can be the estimated weights of the crop, and the exact weights of the apples.

For example, if the user would like to monitor the minimum weight, the load cell is connected to a product 20 that visually represents the minimum weight, as the product 20 that represent the minimum weight changes over time, the weight of the minimum product will be recorded by the device 100, the user can interpret that as the recorded weight representing the crop's minimum weight. Therefore, over time the user can then gauge what would the potential minimum weight of their crop product 20 is if they harvest.

If the user would like to track the average weight of their crop over time, the user will take a sample of the products 20. The average weight of the sample products 20 will be taken and recorded. As the user connects the devices load cell to the product 20 on the plant, the connector is tensioned until the device reads the average weight. As the devices monitors the products 20 weight over time and communicates it to the user, the user can then use the monitored weight to predict or estimate the crops weight in its entirety or portion.

The weights obtained during the recording can represent but are not limited to the actual weights or estimated weights. The weight the device 100 record can be used to interpret a portion of the entirety of the crop.

The weights can be recorded by but not limited to a SD card, on a cloud, hard drive, USB, or any other form of electronic recording.

The weights can be collected over a period of time on the device 100 and recorded on the device 100, and communicated to an external storage location via communication means.

For example, the device 100 collects samples every 1 minute and everyday communicates that data to an external storage location.

If the diameter of the products 20 is being used to estimate the weights, the sensors will monitor the diameter change, the diameter is set at a weight. As the recorded weight is sent to the user, the user can interpret that weight as an estimated whole crop weight via the diameter change of the products 20.

If visual images are being user to determine the product 20 weights, firstly an image of the product 20 is captured, the desired weight is then set by the user for the current visual image of the product 20. As the product 20 changes and is recognised on the visual image, the user can use the estimated weights to present the estimated crop weights.

The user can take the recorded weights from the device and use it to estimate and determine how the overall crop or a part of the crop is functioning. For example, if the device 100 is connected to a product 20 at the average sampled weight, over time as the plant product 20 increases or decreases in weight, it would be estimated that the recorded weight represents a portion or the entirety of the crop.

The information can be used for but is not limited to crop management, irrigation planning, irrigation operations, fertilisers deployment, coverage, spraying regimes, crop dusters, or any other form of crop management that can be undertaken to impact the weight of the products 20.

For example, the user receives weight data and can observe the weight of their products 20 is dropping, in response the user starts to irrigate the crop to increase the product's 20 weight. The user will monitor the recorded weight, within this example, the user installed the load cell to a product 20 and tensioned the connector until the device 100 displayed the average weight of the sample set collected prior. As the weight is changing on the device, the user interprets this as the crop's overall average weight.

Another method that can be used by the user is wherein the plant is restrained so it cannot move.

The plant can be restrained to but not limited to a wall, post, pole, another plant, the ground, or a structure.

Once the plant is restrained this is set as zero weight. This can be undertaken before the plant products 20 are present, preferably when the flowers or buds have started sprouting. As the product 20 changes from a flower into a product 20 the device will start to record the difference from the installation weight to the currently recorded weight.

The devices 100 sensor can also be attached to the plant product 20 at any time, and this is set at weight zero, as the product 20 increases the difference in weight over time from weight zero is recorded and provided to the user. This can be considered differential weight readings, wherein the weight changes over time are used by the users to monitor the product 20 responses.

For example, the user connects the device's 100 sensor to an apple, and the device 100 records 50 grams. 50 grams is recoded as weight zero. As the apples changes weight over time the difference to the 50 grams is recorded. After 8 weeks the apple now weighs 200 grams, it can be considered the apple increased by 150 grams in weight from the initial installation of the device.

Another example can be wherein the user connects the device's sensor to a grape bunch, the sensor ready 450 grams, this is recorded as weight zero. As the weight increases over time, it represents the actual weight increase of the grape bunch in reference to the weight zero 450 grams. 8 weeks later, the device reads 800 grams, this indicates to the user the grape bunch increased to 350 grams in total.

For example, an apple tree is selected to be monitored for apples. The apple branch is connected to the load cell, and the user may restrain the branch, the restraints still allow the branch to move, but reduce the amount of movement. The device 100 is installed, and the weight of the branch is recorded, this will be deemed a zero weight wherein any weight beyond this weight is the product 20 itself. If one or more products 20 grow on the branch the weight can be averaged between the number of products 20, or the user can remove products 20 until a desired number of products 20 are present on the branch such as only one product 20. As the product 20 or products 20 grow on the branch and the weight increases, the device records the weight, and the user can interpret this as the current product 20 weight.

The user can use the collected data from the sensor 25 to predict the future weight in correspondence to environmental conditions, treatments, species, and irrigation practices.

For example, the user forms a database of prior weight data, irrigation management plans, and upcoming environmental conditions to determine the potential product 20 weights. This information can be used to determine yield estimates, and harvest dates.

The device 100 can contain a means to measure the impact of wind on the product 20 affecting the weight recoding. The device 100 can measure the impact of wind with but not limited to a wind vane, accelerometer, force sensor, pressure sensors, movement sensor, laser sensor, gyroscope, irregular load placed on the load cell.

Sensors 25 and devices 100 can be deployed over orchards based on the various soil conditions, wherein the weight data for certain regions of the orchard represent those soil conditions. For example, the user collects a sample of apples from one portion of the orchard known for high yields. The weight data collected over time of the plant products 20 is then applied to that section of the orchard. On the other hand, the other sections of the orchard are treated accordingly. The data is then collated into one pool to estimate the total yield for the orchard, which considers the variations of soil conditions. Though this can be applied to various other variations such as sun light exposure, air, proximity to water, and irrigation variations.

The device 100 can also take readings over a set period of time, the recorded reading is then analysis and can be but not limited to filtering the data, average, removal of outliers, or obtaining the most steady weight over the period of the recording period.

The device 100 can also contain a mechanical means to reduce the amount of impact wind has on the weight recording by introducing a spring, piston, counterweight, electronic motors to counteract wind, or elastic connectors.

The device 100 can contain temperature compensation means wherein the temperature is used to adjust the readings of the sensors.

The device 100 can contain various means to display the data on the enclosure 110, wherein it can be visually observed, or wherein the user can view the information on another device.

### List of Components

The drawings include the following integers.
20 Product
25 Sensor
26 Display
30 Connector
32 Upper connector
35 Connector ring
40 Secondary ring
45 Sensor ring
50 Adjuster
100 Device
110 Enclosure
120 Communication
150 Fixture
200 Fixing

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. A method for real-time measurement of a plant product while it remains attached to a plant, the method comprising:
a. selecting a representative sample of a plant product and measuring its weight to determine a reference weight threshold;
b. attaching a sensor to the plant product, wherein the sensor captures weight data;
c. adjusting the attachment of the sensor to the plant product until the sensor measures a weight within a predefined tolerance of the reference weight threshold; and
d. recording and transmitting the weight data of the plant product to a remote device for monitoring.

2. The sensor of claim 1, wherein the sensor comprises one or more of the following: a load cell, camera, dendrometer, or a combination thereof.

3. The reference weight threshold of claim 1, wherein the threshold is determined based on a minimum, maximum, average, mean, or standard deviation of sample weights.

4. The plant product of claim 1, wherein the plant product comprises a fruit, vegetable, nut, seed, or other agricultural or botanical produce.

5. The sensor of claim 1, wherein the sensor further comprises one or more of a temperature sensor, humidity sensor, optical sensor, infrared sensor, or gas sensor for monitoring environmental and physiological conditions.

6. The sensor of claim 1, wherein the sensor is wirelessly connected to a remote device using Bluetooth, Wi-Fi, LoRa, or another wireless communication protocol for real-time data transmission.

7. The sensor of claim 1, wherein the sensor continuously records weight changes over time and generates a growth trend analysis to predict plant product development.
